(19) United States
(12) Patent Application Publication
(10) Pub. No.: US 2017/0266338 A1
(43) Pub. Date: Sep. 21, 2017

(54) FORMULATION OF FOAM THERMOSENSITIVE HYDROGEL AND METHOD OF MANUFACTURING SAME

(71) Applicants: Chih-Hao Chen, Taoyuan City (TW); Shih-Hsien Chen, Kaohsiung City (TW); Pang-Yun Chou, Taoyuan City (TW); Shih-Hsuan Mao, Taoyuan City (TW); Chien-Hung Liao, Taoyuan City (TW)

(72) Inventors: Chih-Hao Chen, Taoyuan City (TW); Shih-Hsien Chen, Kaohsiung City (TW); Pang-Yun Chou, Taoyuan City (TW); Shih-Hsuan Mao, Taoyuan City (TW); Chien-Hung Liao, Taoyuan City (TW)

(21) Appl. No.: 15/072,369

(22) Filed: Mar. 17, 2016

(51) Int. Cl. A61L 26/00

(52) U.S. Cl. CPC A61L 26/0014; A61L 26/008; A61L 26/0085; A61L 26/0061; A61L 2300/424; A61L 2400/06

(57) ABSTRACT

A formulation of foam thermosensitive hydrogel includes a thermosensitive hydrogel including synthetic polymeric material. The thermosensitive hydrogel has properties of changing phase from liquid to solid in high temperature and changing phase from solid to liquid in low temperature. The foam thermosensitive hydrogel having a predetermined surface tension is formed after mixing the thermosensitive hydrogel with air. The foam thermosensitive hydrogel has properties of increased volume and being semisolid. After injecting the foam thermosensitive hydrogel into a human body, the foam thermosensitive hydrogel changes phase form liquid to solid after temperature rises above LCST. The foam thermosensitive hydrogel injected into the human body becomes a solid, physical barrier between an injured site and surrounding tissue, thereby preventing adhesion from forming in the human body. A method of manufacturing foam thermosensitive hydrogel is also provided.

### BACKGROUND OF THE INVENTION

1. Field of the Invention

The invention relates to anti-adhesion formulation and more particularly to a formulation of foam thermosensitive hydrogel and method of manufacturing same so that no more excessive foreign objects are injected in surgery and the thermosensitive hydrogel does not move from a desired site of the human body to other undesired sites after the injection.

2. Description of Related Art

Physicians are bothered by adhesion formed on the tissue after surgery. Injury can cause inflammation which in turn absorbs fibroblasts of surrounding tissue for healing the injured site. However, excessive fibrous tissues are created to form adhesion between the injured site and the surrounding tissue. Adhesion may form on the abdomen for more than 93% of patients after surgery. The adhesion may cause chronic abdominal pain, pelvic cavity pain, permanent infertility, and/or intestine blockage in a serious case. Adhesion may form after tendon surgery. Typically, adhesion may form on the tendon and the surrounding tissue to limit pivotal movement of joints.

Conventionally, thermosensitive hydrogel is employed to synthesize a three-part copolymer such as hydrophobic polymer-hydrophilic polymer-hydrophobic polymer or hydrophilic polymer-hydrophobic polymer-hydrophilic polymer. Natural polymeric materials do not have property of being thermosensitive. Thus, they are required to modify or synthesize so as to be thermosensitive. Thus, very few natural polymeric materials having thermosensitive property are employed in applications of anti-adhesion. For example, only chitosan and methyl cellulose are employed on abdomen for anti-adhesion. Currently, thermosensitive hydrogel is employed in applications of anti-adhesion and a large amount of hydrogel is required. And in turn, excessive foreign objects are injected. Further, the hydrogel may move from a desired site of the human body to other undesired sites after the injection.

Thus, the need for improvement still exists.

### SUMMARY OF THE INVENTION

It is therefore one object of the invention to provide a formulation of foam thermosensitive hydrogel comprising a thermosensitive hydrogel including synthetic polymeric material; wherein the thermosensitive hydrogel has properties of changing phase from liquid to solid in high temperature and changing phase from solid to liquid in low temperature; wherein the foam thermosensitive hydrogel having a predetermined surface tension is formed after mixing the thermosensitive hydrogel with air; wherein the foam thermosensitive hydrogel has properties of increased volume and being semisolid; wherein after injecting the foam thermosensitive hydrogel into a human body, the foam thermosensitive hydrogel changes phase form liquid to solid after temperature rises above LCST; and wherein the foam thermosensitive hydrogel injected into the human body becomes a solid, physical barrier between an injured site and surrounding tissue, thereby preventing adhesion from forming in the human body.

It is therefore another object of the invention to provide a method of manufacturing foam thermosensitive hydrogel comprising the steps of adding saline to synthetic polymeric material in a reagent bottle to prepare a solution of water at 1-45% concentration; stirring and keeping the reagent bottle at 4° C. mixing same, and forming the solution to prepare thermosensitive hydrogel in the reagent bottle; and taking the thermosensitive hydrogel out of the reagent bottle to mix with air in a ratio of 1:20 to 20:1, thereby preparing foam thermosensitive hydrogel having tiny bubbles.

The above and other objects, features and advantages of the invention will become apparent from the following detailed description taken with the accompanying drawings.

### BRIEF DESCRIPTION OF THE DRAWINGS

FIG. 1 is a photograph showing from left to right typical thermosensitive hydrogel in a first reagent bottle at 25° C. thermosensitive hydrogel having 20% (w/v) foam concentration in a second reagent bottle at 25° C. and mixed with air, the typical thermosensitive hydrogel in a first reagent bottle after being heated to 37° C., and the thermosensitive hydrogel having 20% (w/v) foam concentration in the second reagent bottle at 37° C. after mixed with the air and heated to 37° C. according to the invention;

FIG. 2 shows ten images of foam thermosensitive hydrogel having different concentrations of PNIPAM according to the invention;

FIG. 3 is a chart showing bubble size versus concentration of PNIPAM for the foam thermosensitive hydrogel having different concentrations of PNIPAM according to the invention;

FIG. 4A is a chart showing volume change versus concentration of PNIPAM for the foam thermosensitive hydrogel having different concentrations of PNIPAM after mixing with air according to the invention;

FIG. 4B is a chart showing volume versus time for the foam thermosensitive hydrogel having different concentrations of PNIPAM according to the invention;

FIG. 5 contains eight images each showing the foam thermosensitive hydrogel having a different concentration of PNIPAM at 25° C. for observing volume change and appearance change according to the invention;

FIG. 6 contains six images taken by camera (top row) and optical microscope (bottom row) four weeks after abdominal surgery and the images show adhesion (black arrows) and hematoxylin and eosin staining of the tissue section (bottom row) in which C means cecum and AW means abdominal wall according to the invention;

FIG. 7A is a table showing scores recorded based on images of gross view shown in FIG. 6 according to the invention;

FIG. 7B is a table showing scores recorded based on images of hematoxylin and eosin staining of the tissue section shown in FIG. 6 according to the invention; and

FIG. 8 is a flowchart illustrating a method of manufacturing foam thermosensitive hydrogel according to the invention.

### DETAILED DESCRIPTION OF THE INVENTION

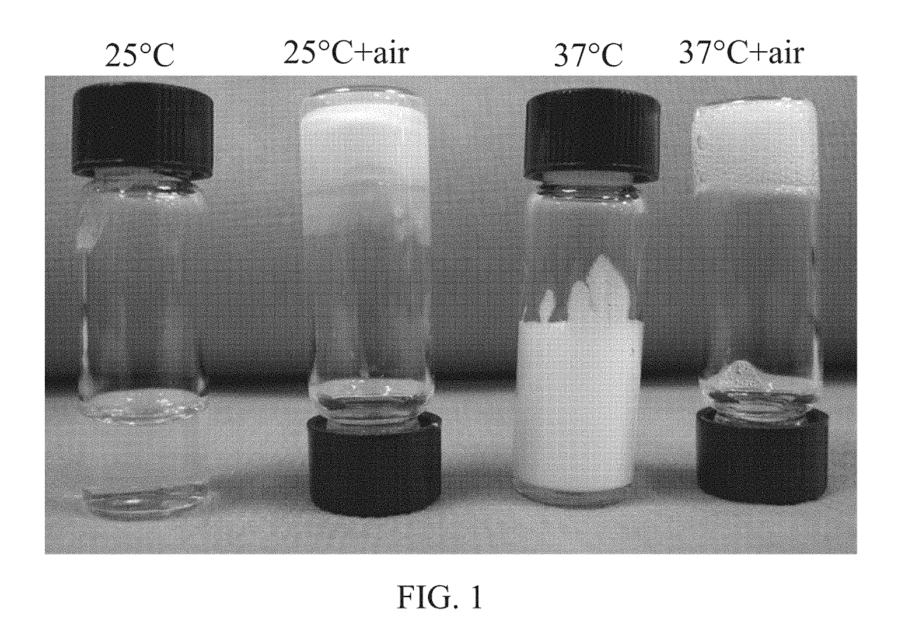

Referring to FIGS. 1 to 7B, a formulation of foam thermosensitive hydrogel in accordance with the invention is shown.

In FIG. 1, typical thermosensitive hydrogel is poured into the leftmost reagent bottle and thermosensitive hydrogel having 20% (weight to volume (w/v)) foam concentration is poured into the reagent bottle next to the leftmost reagent bottle and together they are placed in room temperature (i.e., 25° C.) with a first photograph of the two reagent bottles is taken. Further, they are heated to 37° C. for ten minutes with a second photograph of the two reagent bottles is taken. The first and second photographs are combined to create a photograph as shown in FIG. 1. Note that the reagent bottles containing thermosensitive hydrogel having 20% (w/v) foam concentration are upside down.

In room temperature (i.e., 25° C.) it is observed from the left two reagent bottles that the typical thermosensitive hydrogel is colorless, transparent and adapted to flow as liquid, and there are many tiny bubbles in the thermosensitive hydrogel having 20% (w/v) foam concentration. Further, the thermosensitive hydrogel having 20% (w/v) foam concentration is white because a beam of light passing through is interfered by the bubbles. Furthermore, the thermosensitive hydrogel having 20% (w/v) foam concentration has property as a gel due to surface tension so that the reagent bottle containing the thermosensitive hydrogel having 20% (w/v) foam concentration can be placed upside down.

In temperature of 37° C. it is observed from the right two reagent bottles that both the typical thermosensitive hydrogel and the thermosensitive hydrogel having 20% (w/v) foam concentration are white because they have property as a gel. Further, the bubbles in the reagent bottle containing the thermosensitive hydrogel having 20% (w/v) foam concentration are visible. Reasons of the typical thermosensitive hydrogel becoming white are detailed below. After heating the typical thermosensitive hydrogel to a temperature greater than lower critical solution temperature (LOST) (i.e., about 32° C.), molecules of hydrophobic Poly(N-isopropylacrylamide) (PNIPAM) therein cross-link to form gel which interferes the passing of beam of light. As a result, the typical thermosensitive hydrogel becomes white and opaque.

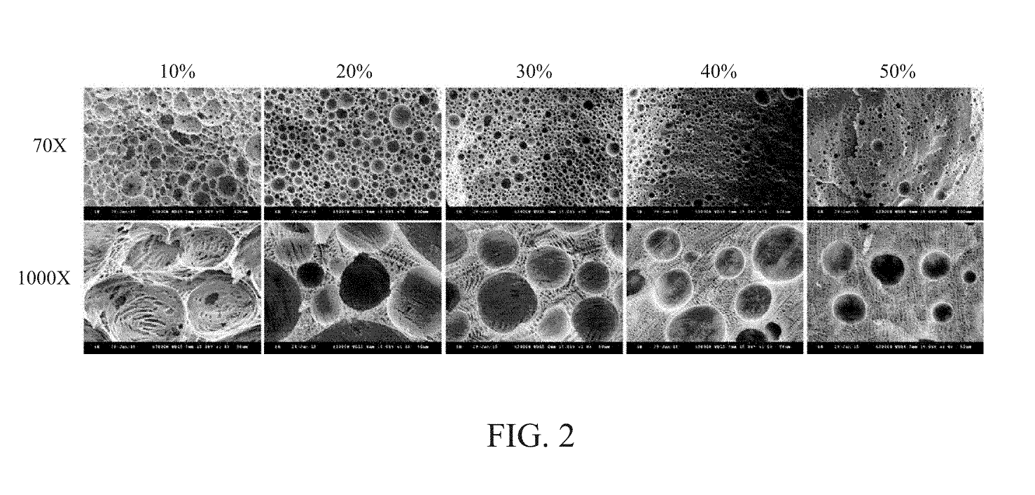

In FIG. 2, ten images taken by a scanning electron microscope are shown. Bubble sizes in the foam hydrogel become smaller as concentration of PNIPAM increases. Many larger holes are formed on the bubbles when PNIPAM has lower concentration. This is because PNIPAM having lower concentration cannot fill the bubbles.

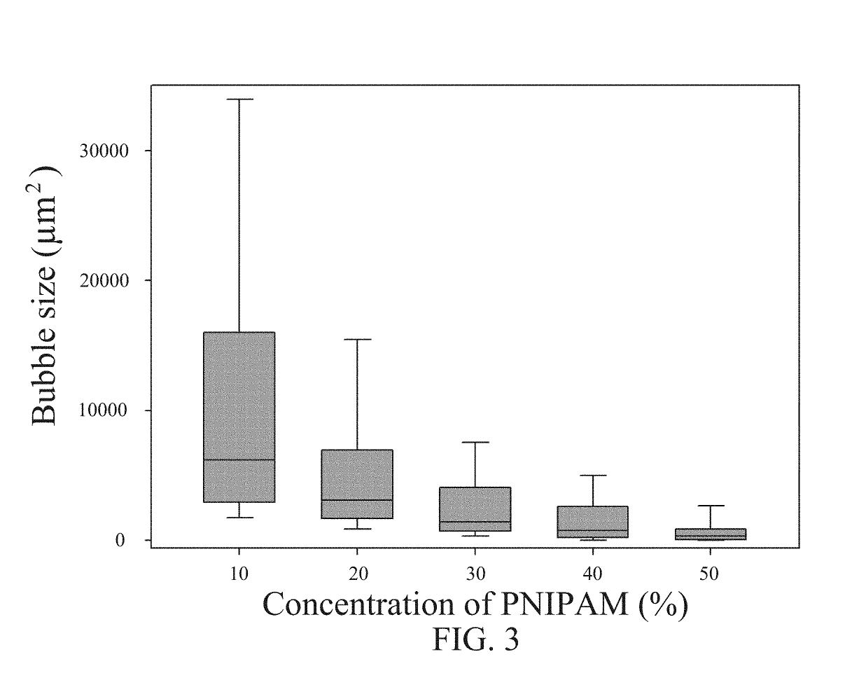

In FIG. 3, it can be seen that bubble sizes decrease as concentration of PNIPAM increases. Further, distribution range decreases. Molecules increase when PNIPAM having higher concentration is mixed with gas. And in turn, both adhesion and surface tension increase. As a result, bubble sizes become smaller.

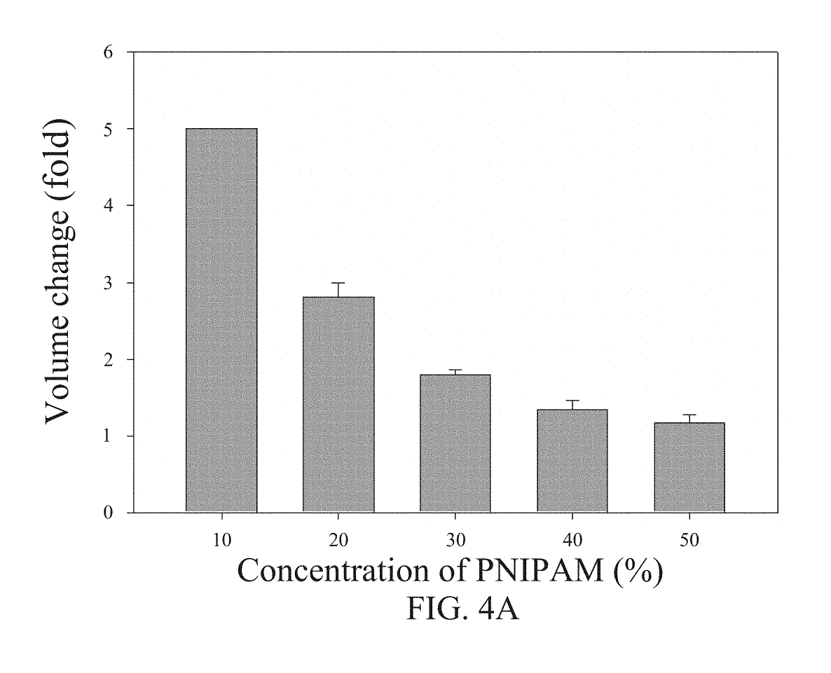

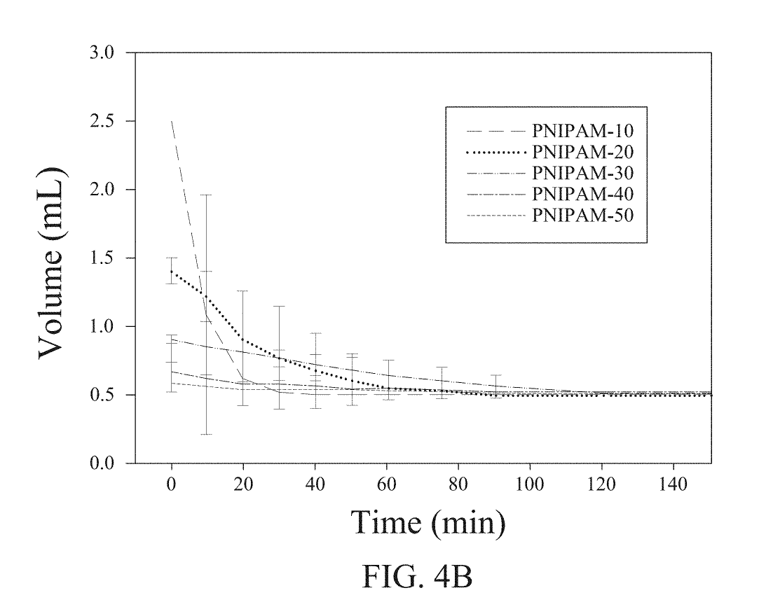

In FIG. 4A, it can seen that volume change decreases as concentration of PNIPAM increases. Further, distribution range decreases. After increasing concentration of PNIPAM, volume change decreases in comparison with volume of hydrogel containing no bubble. This is because bubble sizes in the hydrogel having high concentration of PNIPAM become smaller. And in turn, less gas can be mixed. To the contrary, more large bubbles can be mixed with hydrogel having lower concentration of PNIPAM. And in turn, volume change increases. For convenience in future use, it is desirable to know how long will bubbles last in room temperature.

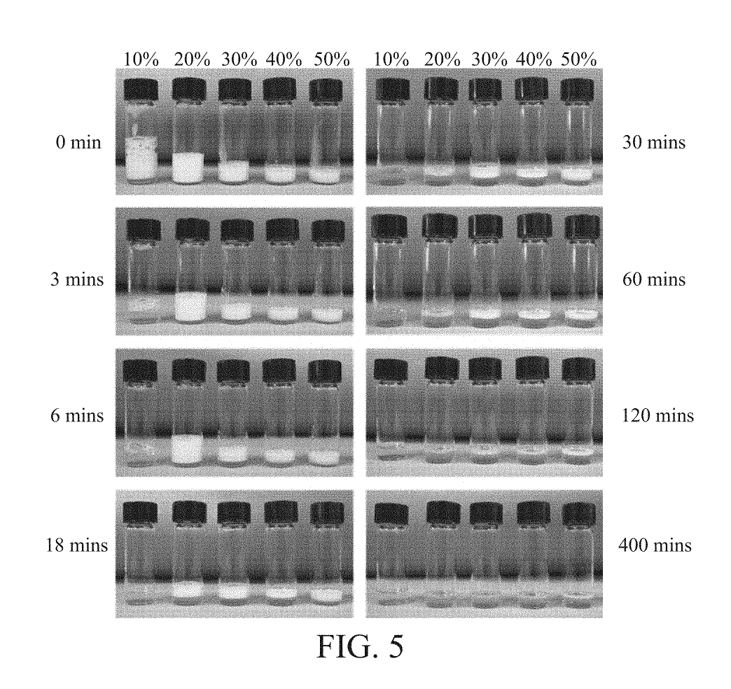

In FIGS. 4B and 5, it can be seen that foam thermosensitive hydrogel having 10% concentration of PNIPAM can last 20 minutes in a bubble state, foam thermosensitive hydrogel having 20% concentration of PNIPAM can last 60 minutes in a bubble state, and foam thermosensitive hydrogel having 30% concentration of PNIPAM can last 120 minutes in a bubble state. It is concluded that foam thermosensitive hydrogel having higher concentration of PNIPAM can last more time in a bubble state.

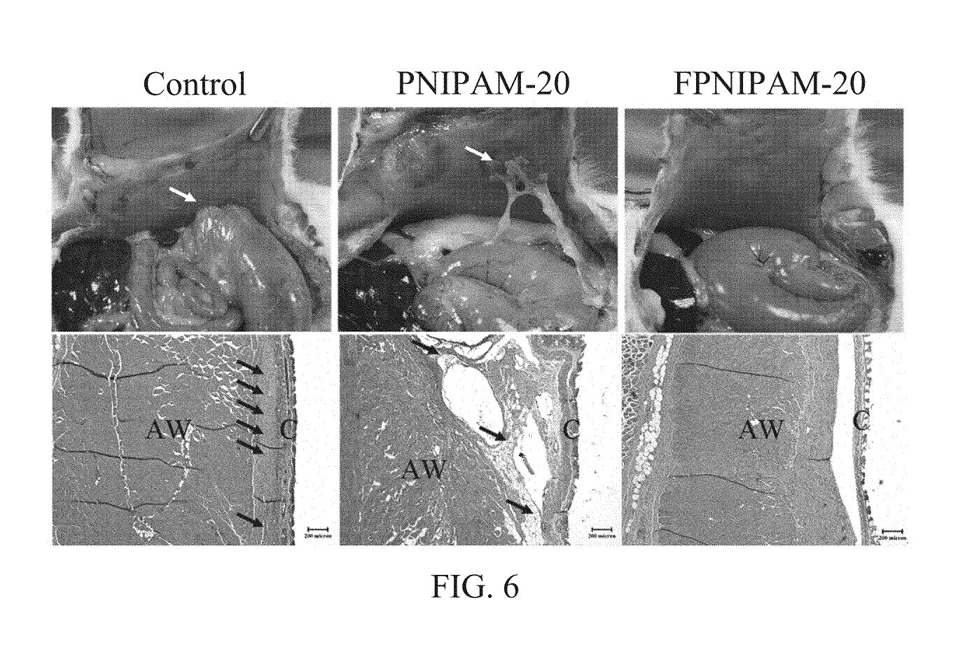

Foam thermosensitive hydrogel having 40% concentration of PNIPAM (i.e., FPNIPAM-40 (Foamy Poly(N-isopropylacrylamide)-40) and thermosensitive hydrogel having 50% concentration of PNIPAM (i.e., FPNIPAM-50) are eliminated initially in consideration of convenience in future use. Also, small volume increase of foam thermosensitive hydrogel cannot achieve the purpose of adhesion separation because small volume of hydrogel does not occupy large space. Foam thermosensitive hydrogel having 10% concentration of PNIPAM (i.e., PNIPAM-10) lasts for a very short time in a bubble state and in turn, it may limit time in use. As to foam thermosensitive hydrogel having 20% concentration of PNIPAM (i.e., FPNIPAM-20) and foam thermosensitive hydrogel having 30% concentration of PNIPAM (i.e., FPNIPAM-30), both have volume change of 20%. Less PNIPAM is contained in the foam thermosensitive hydrogel and in turn it decreases foreign objects injected. Therefore, foam thermosensitive hydrogel having 20% concentration of PNIPAM is defined as FPNIPAM-20 for future experiment as discussed in detail in FIG. 6.

In FIG. 6, it shows six images taken by camera (top row) and optical microscope (bottom row) four weeks after abdominal surgery and the images show adhesion and hematoxylin and eosin staining of the tissue section (bottom row). As shown, adhesion does not form on abdominal wall (AW) and cecum (C) or surrounding tissue in the FPNIPAM-20 as experiment set. As to control set, adhesion forms on AW and C or surrounding tissue. The adhesion is a result of injured site surrounded by large fibrous tissue. Thus, strong force is required to separate AW from C. As to FPNIPAM-20, injured portion of AW is connected to loose fat and fibrous tissue. But a small force is still required to break the connection. In view of the images, FPNIPAM-20 has good anti-adhesion effect and therefore can prevent adhesion between AW and C (or surrounding tissue).

As to the other two sets for prevention of AW adhesion, it is found that there is severe adhesion on AW in the control set. As to the FPNIPAM-20, injured portion of AW is connected to loose fibrous tissue.

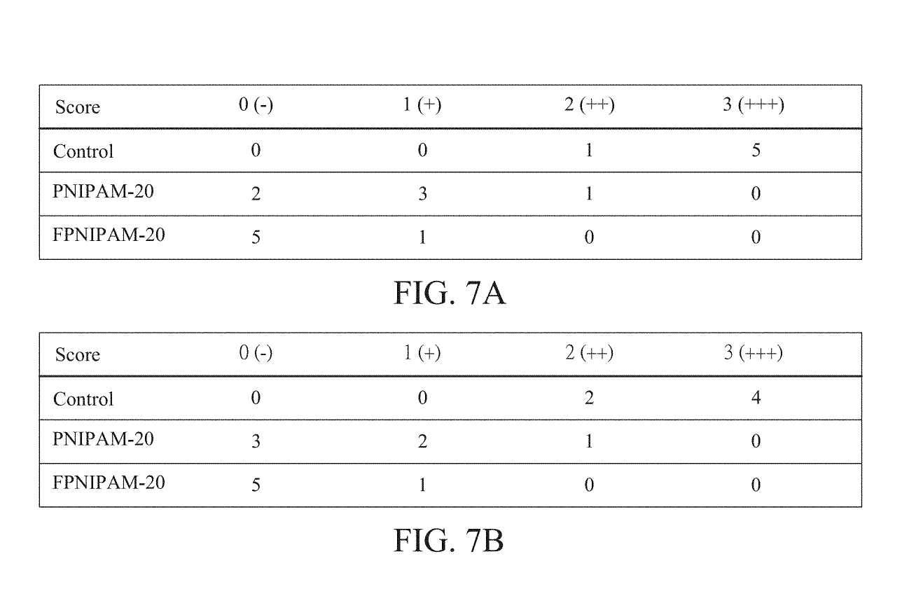

For quantitatively evaluating anti-adhesion effect of different sets, we took several images of hematoxylin and eosin staining of the tissue section for observation in which lower score means less adhesion as discussed in detail in FIG. 7A.

In FIG. 7A, this table is tabulated by a physician not participating in the experiment after reviewing images shown in FIG. 6. Score 0 means no adhesion. Score 1 means mild adhesion. Score 2 means moderate adhesion. Score 3 means severe adhesion. Each set takes six participating SD (Spraque Dawley) rats as samples. For control set, there is one rat having a score of 2 (i.e., moderate adhesion) and there are five rats having a score of 3 (i.e., severe adhesion). For PNIPAM-20 set, there are two rats having a score of 0 (i.e., no adhesion), there are three rats having a score of 1 (i.e., mild adhesion), and there is one rat having a score of 2 (i.e., moderate adhesion). For FPNIPAM-20, there are five rats having a score of 0 (i.e., no adhesion) and there is one rat having a score of 1 (i.e., mild adhesion).

In FIG. 7B, this table is tabulated by a physician not participating in the experiment after reviewing images of hematoxylin and eosin staining of the tissue section shown in FIG. 6. Score 0 means no adhesion. Score 1 means mild adhesion. Score 2 means moderate adhesion. Score 3 means severe adhesion. Each set takes six participating SD rats as samples. For control set, there is two rats having a score of 2 (i.e., moderate adhesion) and there are four rats having a score of 3 (i.e., severe adhesion). For PNIPAM-20 set, there are three rats having a score of 0 (i.e., no adhesion), there are two rats having a score of 1 (i.e., mild adhesion), and there is one rat having a score of 2 (i.e., moderate adhesion). For FPNIPAM-20, there are five rats having a score of 0 (i.e., no adhesion) and there is one rat having a score of 1 (i.e., mild adhesion).

As shown in FIGS. 7A and 7B, it is found that in FPNIPAM-20 as anti-adhesion prevention set, most rats have score of 0 and it means the probability of forming adhesion is very low. Further, in PNIPAM-20 set, some mice have score of 1 or 2 and it means the probability of forming adhesion is higher. Furthermore, in the control set, most rats have score of 3 and it means the probability of forming adhesion is highest.

In views of the above discussion, FPANIPAM-20 is more effective than PNIPAM-20 and PNIPAM-20 is more effective than control set in terms of anti-adhesion effect. It is concluded that FPNIPAM-20 has a great potential as a candidate for preventing adhesion from forming on abdominal wall of human body.

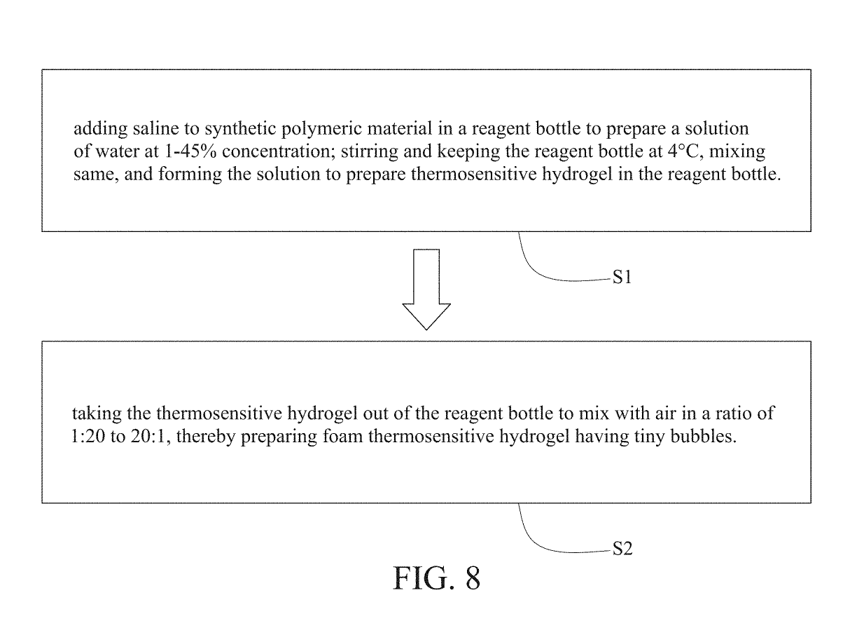

Referring to FIG. 8, a formulation and method of manufacturing foam thermosensitive hydrogel in accordance with the invention is illustrated. The method comprises the following steps:

S1: Saline is added to synthetic polymeric material in a reagent bottle to prepare a solution of water at 1-45% concentration. Next, the reagent bottle is stirring and keeping at 4° C. and mixed. Thermosensitive hydrogel is prepared in the reagent bottle after the solution is formed.

S2: The thermosensitive hydrogel is taken out of the reagent bottle to mix with air in a ratio of 1:20 to 20:1. A foam thermosensitive hydrogel having tiny bubbles is prepared.

Preferably, the synthetic polymeric material is PNIPAM, polyoxyethylene-polyoxypropylene-polyoxyethylene, polycaprolactone-polyethylene glycol (PCL-PEG), polycaprolactone-polyethylene glycol-polycaprolactone (PCL-PEG-PCL), polylactic acid-polyethylene glycol (PLA-PEG), polylactic glycolic acid-polyethylene glycol (PLGA-PEG), polylactic glycolic acid-polyethylene glycol-polylactic glycolic acid (PLGA-PEG-PLGA), polycaprolactone-co-lactide-polyethylene glycol (PCLA-PEG), polycaprolactone-co-lactide-polyethylene glycol-polycaprolactone-co-lactide (PCLA-PEG-PCLA), polycaprolactone, polylactic glycolic acid, polyethylene glycol, polylactic acid, chitosan, alginate, hyaluronic acid, collagen, gelatin, or cellulose.

Preferably, the mixing of the thermosensitive hydrogel with the air is done by drawing the thermosensitive hydrogel into a first syringe, drawing the air into a second syringe, interconnecting to the first and second syringes with an adapter, and repeatedly pushing the air into the first syringe to mix with the thermosensitive hydrogel to form a half-finished mixture and pushing the half-finished mixture into the second syringe for 20 times until a foam thermosensitive hydrogel having tiny bubbles is prepared.

Preferably, the mixing of the thermosensitive hydrogel with the air is done by pouring the thermosensitive hydrogel into a reagent bottle, and squeezing the bellows top of the reagent bottle a predetermined number of times to prepare a foam thermosensitive hydrogel having tiny bubbles. The reagent bottle is commercially available.

Preferably, the foam thermosensitive hydrogel has properties of changing phase from liquid to solid and changing phase from solid to liquid.

Preferably, the phase change temperature is in the range from 1° C. to 50° C.

Preferably, the temperature of changing phase from liquid to solid is in the range from 1° C. to 40° C.

Preferably, the temperature of changing phase from solid to liquid is in the range from 20° C. to 50° C.

Preferably, bubble size of the foam thermosensitive hydrogel is in the range from 10 nm to 5,000,000 nm.

The invention has the following characteristics and advantages:

Percentage of thermosensitive hydrogel in foam thermosensitive hydrogel is less in terms of volume. Less gel is contained in the foam thermosensitive hydrogel and in turn it decreases foreign objects injected. Surface tension of the foam thermosensitive hydrogel can prevent it from flowing to other parts. The foam thermosensitive hydrogel injected into the human body becomes a solid, physical barrier between the injured site and the surrounding tissue, thereby preventing adhesion from forming in the human body.

The thermosensitive hydrogel contains synthetic polymeric material. The thermosensitive hydrogel has properties of changing phase from liquid to solid in high temperature and changing phase from solid to liquid in low temperature. The foam thermosensitive hydrogel having a predetermined surface tension is formed after mixing thermosensitive hydrogel with air. The foam thermosensitive hydrogel has properties of increased volume and being semisolid. After injecting the foam thermosensitive hydrogel into a human body, it may change phase form liquid to solid after temperature rises above LCST.

Preferably, the foam thermosensitive hydrogel can be injected into abdomen, thoracic cavity, pelvic cavity, tendon, nerves, or spines in surgery.

Preferably, the foam thermosensitive hydrogel solution has water at 1-45% concentration.

Preferably, the phase change temperature is in the range from 1° C. to 50° C. Preferably, the LCST is in the range from 4° C. to 36° C.

Preferably, the synthetic polymeric material is PNIPAM, polyoxyethylene-polyoxypropylene-polyoxyethylene, polycaprolactone-polyethylene glycol (PCL-PEG), polycaprolactone-polyethylene glycol-polycaprolactone (PCL-PEG-PCL), polylactic acid-polyethylene glycol (PLA-PEG), polylactic glycolic acid-polyethylene glycol (PLGA-PEG), polylactic glycolic acid-polyethylene glycol-polylactic glycolic acid (PLGA-PEG-PLGA), polycaprolactone-co-la tide-polyethylene glycol (PCLA-PEG), polycaprolactone-co-lactide-polyethylene glycol-polycaprolactone-co-lactide (PCLA-PEG-PCLA), polycaprolactone, polylactic glycolic acid, polyethylene glycol, polylactic acid, chitosan, alginate, hyaluronic acid, collagen, gelatin, or cellulose.

While the invention has been described in terms of preferred embodiments, those skilled in the art will recognize that the invention can be practiced with modifications within the spirit and scope of the appended claims.

What is claimed is:

1. A formulation of foam thermosensitive hydrogel comprising:
a thermosensitive hydrogel including synthetic polymeric material;
wherein the thermosensitive hydrogel has properties of changing phase from liquid to solid in high temperature and changing phase from solid to liquid in low temperature;
wherein the foam thermosensitive hydrogel having a predetermined surface tension is formed after mixing the thermosensitive hydrogel with air;
wherein the foam thermosensitive hydrogel has properties of increased volume and being semisolid;
wherein after injecting the foam thermosensitive hydrogel into a human body, the foam thermosensitive hydrogel changes phase form liquid to solid after temperature rises above LCST; and
wherein the foam thermosensitive hydrogel injected into the human body becomes a solid, physical barrier between an injured site and surrounding tissue, thereby preventing adhesion from forming in the human body.

2. The formulation of foam thermosensitive hydrogel of claim 1, wherein the foam thermosensitive hydrogel is configured to inject into abdomen, thoracic cavity, pelvic cavity, tendon, nerves, or spines in surgery.

3. The formulation of foam thermosensitive hydrogel of claim 1, wherein the foam thermosensitive hydrogel solution has water at 1-45% concentration.

4. The formulation of foam thermosensitive hydrogel of claim 1, wherein the phase change temperature is in the range from 1° C. to 50° C.

5. The formulation of foam thermosensitive hydrogel of claim 1, wherein the LCST is in the range from 4° C. to 36° C.

6. The formulation of foam thermosensitive hydrogel of claim 1, wherein the synthetic polymeric material is PNIPAM, polyoxyethylene-polyoxypropylene-polyoxyethylene; polycaprolactone-polyethylene glycol (PCL-PEG), polycaprolactone-polyethylene glycol-polycaprolactone (PCL-PEG-PCL), polylactic acid-polyethylene glycol (PLA-PEG); polylactic glycolic acid-polyethylene glycol (PLGA-PEG), polylactic glycolic acid-polyethylene glycol-polylactic glycolic acid (PLGA-PEG-PLGA), polycaprolactone-co-lactide-polyethylene glycol (PCLA-PEG), polycaprolactone-co-lactide-polyethylene glycol-polycaprolactone-co-lactide (PCLA-PEG-PCLA), polycaprolactone, polylactic glycolic acid, polyethylene glycol, polylactic acid, chitosan, alginate, hyaluronic acid, collagen, gelatin, or cellulose.

7. A method of manufacturing foam thermosensitive hydrogel comprising the steps of:
adding saline to synthetic polymeric material in a reagent bottle to prepare a solution of water at 1-45% concentration;
stirring and keeping the reagent bottle at 4° C., mixing same, and forming the solution to prepare thermosensitive hydrogel in the reagent bottle; and
taking the thermosensitive hydrogel out of the reagent bottle to mix with air in a ratio of 1:20 to 20:1, thereby preparing foam thermosensitive hydrogel having tiny bubbles.

8. The method of manufacturing foam thermosensitive hydrogel of claim 7, wherein the synthetic polymeric material PNIPAM, polyoxyethylene-polyoxypropylene-polyoxyethylene, polycaprolactone-polyethylene glycol (PCL-PEG), polycaprolactone-polyethylene glycol-polycaprolactone (PCL-PEG-PCL), polylactic acid-polyethylene glycol (PLA-PEG), polylactic glycolic acid-polyethylene glycol (PLGA-PEG), polylactic glycolic acid-polyethylene glycol-polylactic glycolic acid (PLGA-PEG-PLGA), polycaprolactone-co-lactide-polyethylene glycol (PCLA-PEG), polycaprolactone-co-lactide-polyethylene glycol-polycaprolactone-co-lactide (PCLA-PEG-PCLA), polycaprolactone, polylactic glycolic acid, polyethylene glycol, polylactic acid, chitosan, alginate, hyaluronic acid, collagen, gelatin, or cellulose.

9. The method of manufacturing foam thermosensitive hydrogel of claim 7, wherein the step of mixing thermosensitive hydrogel with air is done by drawing the thermosensitive hydrogel into a first syringe, drawing the air into a second syringe, interconnecting to the first and second syringes with an adapter, and repeatedly pushing the air into the first syringe to mix with the thermosensitive hydrogel to form a half-finished mixture and pushing the half-finished mixture into the second syringe for 20 times until the foam thermosensitive hydrogel is prepared.

10. The method of manufacturing foam thermosensitive hydrogel of claim 7, wherein the mixing of the thermosensitive hydrogel with the air is done by pouring the thermosensitive hydrogel into a reagent bottle, and squeezing bellows top of the reagent bottle a predetermined number of times to prepare the foam thermosensitive hydrogel.

11. The method of manufacturing foam thermosensitive hydrogel of claim 7, wherein the foam thermosensitive hydrogel has properties of changing phase from liquid to solid and changing phase from solid to liquid.

12. The method of manufacturing foam thermosensitive hydrogel of claim 11, wherein the phase change temperature is in the range from 1° C. to 50° C.

13. The method of manufacturing foam thermosensitive hydrogel of claim 12, wherein the temperature of changing phase from liquid to solid is in the range from 1° C. to 40° C., and the temperature of changing phase from solid to liquid is in the range from 20° C. to 50° C.

14. The method of manufacturing foam thermosensitive hydrogel of claim 7, wherein bubble size of the foam thermosensitive hydrogel is in the range from 10 nm to 5,000,000 nm.
